(19) United States
(12) Patent Application Publication
(10) Pub. No.: US 2008/0113955 A1
(43) Pub. Date: May 15, 2008

(54) TREATMENT OF INFANT HYPERBILIRUBINEMIA USING LOW DOSAGES OF STANNSOPORFIN

(76) Inventors: Benjamin LEVINSON, Montgomery, NJ (US); Simon Tulloch, Chester Springs, PA (US)

Correspondence Address: MORRISON & FOERSTER LLP, 755 PAGE MILL RD, PALO ALTO, CA, 94304-1018, US

(21) Appl. No.: 11/867,581

(22) Filed: Oct. 4, 2007

Related U.S. Application Data
(60) Provisional application No. 60/849,509, filed on Oct. 4, 2006.

(51) Int. Cl. A61K 31/555; A61P 43/00

(52) U.S. Cl. 514/185

(57) ABSTRACT

Methods of treating infant hyperbilirubinemia using stannsoporfin are disclosed. The methods of the invention permit treatment of various patient populations at lower doses of stannsoporfin than previously believed effective, providing advantages in therapeutic administration.

### CROSS REFERENCE TO RELATED APPLICATIONS

This application claims priority benefit of U.S. Provisional Patent Application No. 60/849,509, filed on Oct. 4, 2006. The content of that application is hereby incorporated by reference herein in its entirety.

### TECHNICAL FIELD

This invention pertains to methods for treating infant hyperbilirubinemia using stannsoporfin (tin (IV) mesoporphyrin IX dichloride) at drug doses significantly lower than those previously believed to be efficacious.

### BACKGROUND

Infant hyperbilirubinemia (also known as infant jaundice or neonatal hyperbilirubinemia) occurs in a newborn when the liver is unable to conjugate bilirubin so it can be excreted at a rate commensurate with bilirubin formation. Bilirubin comes from the release of heme as part of the physiological conversion from fetal to adult hemoglobin at birth. The enzyme heme oxygenase oxidizes heme to biliverdin; the enzyme biliverdin reductase then reduces the biliverdin to bilirubin. Bilirubin at high serum levels is a neurotoxic substance. In adult humans, the liver rapidly converts bilirubin into a conjugated, excretable form. In newborn humans, however, the liver is still developing, and uptake and conjugation by the liver is not as efficient as in adults. Additionally, hemolysis may be taking place at a greater relative rate than in adults. All of these factors can lead to excessive bilirubin in the infant. For some infants, high serum levels of bilirubin can have detrimental physiological consequences. Bilirubin is yellow, and infants with excess bilirubin appear jaundiced, having a yellow tinge to their skin and to the whites of their eyes.

Infants who have highly elevated serum levels of bilirubin are at risk of developing kernicterus, a rare but potentially devastating neurological disorder which can result in severe life-long disabilities and complications such as athetosis, hearing loss, vision problems, and dental problems. (See Centers for Disease Control and Prevention World-Wide-Web.cdc.gov/ncbddd/dd/kernicterus.htm.) Accordingly, infants should be carefully monitored after birth, and therapeutic intervention should be commenced if an infant's bilirubin level is excessive. The American Academy of Pediatrics has published a Clinical Practice Guideline for evaluating newborns for hyperbilirubinemia and treating at-risk newborns; see Pediatrics 114:297-316 (2004). Due to rising health-care and hospitalization costs, seemingly healthy newborns and their mothers are discharged rapidly, sometimes as quickly as 24 to 48 hours after birth. However, it is believed that this practice may have contributed to an increase in cases of kernicterus, which had been virtually eliminated from developed countries; see Hansen TWR, Acta Paediatr. 89:1155-1157 (2000)). Because early discharge can delay the detection of jaundice and hyperbilirubinemia in infants, effective means of treating hyperbilirubinemia rapidly are desirable. The unique medical status of the newborn also requires that any means of treatment be as safe as possible, as side effects that are tolerable in adults may be completely unacceptable in neonates.

Currently approved and commonly used treatments for hyperbilirubinemia include phototherapy and exchange transfusion. Phototherapy involves irradiating the newborn with light in the 430 to 490 nm range (blue light). The light converts bilirubin into lumirubin and photobilirubin, which are more readily excreted by the infant, and thus can result in a reduction of bilirubin levels.

In severe cases, phototherapy is insufficient to reduce bilirubin levels, and an exchange transfusion must be performed. An exchange transfusion is a medical procedure where serum bilirubin is reduced by gradual removal of infant blood volume and replacement with blood having low bilirubin levels. This procedure carries certain risks; death (about 3 for every 1000 exchange transfusions) have been reported, as well as cardiac arrhythmias, and complications (such as thrombosis and vasospasm) can occur in as many as 1 in 20 exchange transfusions (see Pediatrics 114:297-316 (2004)).

Accordingly, a safe and effective alternative to exchange transfusion would offer significant benefits in developed countries. Additionally, in developing countries, phototherapy and exchange transfusion may not be readily available.

Stannsoporfin, or tin (IV) mesoporphyrin IX dichloride, is an inhibitor of the enzyme heme oxygenase. Stannsoporfin has been proposed for therapeutic use in infant hyperbilirubinemia (U.S. Pat. No. 4,657,902; U.S. Pat. No. 4,668,670; WO 94/28906). Syntheses of stannsoporfin are disclosed in U.S. Pat. No. 6,818,763, U.S. Patent Application Publication No. 2004/0210048, U.S. Patent Application Publication No. 2006/0222668, U.S. Provisional Patent Application No. 60/849,641, filed Oct. 4, 2006, U.S. Provisional Patent Application No. 60/904,601, filed Feb. 28, 2007, U.S. patent application Ser. No. ______ (Attorney Docket No. 606952000100) filed on Oct. 4, 2007, and International (Patent Cooperation Treaty) patent application Ser. No. ______ (Attorney Docket No. 606952000140) filed on Oct. 4, 2007. Additional methods of using stannsoporfin are disclosed in U.S. Pat. No. 4,692,440 (to increase the rate of heme excretion), WO 89/02269 (to counteract the toxicity of cancer therapy), U.S. Pat. No. 4,782,049 (to treat psoriasis) and other publications.

Various dosages of stannsoporfin have been proposed and studied in different infant (and adult) populations. Doses of stannsoporfin ranging from 1 umol per kg birthweight to 6 umol/kg birthweight (0.75 mg/kg birthweight to 4.5 mg/kg birthweight) in pre-term (premature) infants were studied by Valaes et al., Pediatrics 93:1-11 (1994). A study in Greece showed that a single dose of stannsoporfin of 6 umol/kg birthweight (4.5 mg/kg birthweight) could avoid the need for phototherapy in jaundiced infants; see Kappas et al., Pediatrics, 95:468-474 (1995).

It has now been shown by the inventors that stannsoporfin can be employed therapeutically in infants at doses much lower than those previously used, a development which will enhance the utility of stannsoporfin therapy significantly, as physicians are naturally reluctant to administer any pharmaceutical to a newborn, and a reduction in dosage is often associated with a reduction in complications. Stannsoporfin also offers the advantage of preventing the need for further phototherapy or exchange transfusion once the initial therapy has been discontinued. This further need is termed “rebound” hyperbilirubinemia, and is found in about 20% of babies who have hemolytic disease.

### DISCLOSURE OF THE INVENTION

The current invention embraces, in certain aspects, use of stannsoporfin at dosages significantly lower than those dosages previously shown to be effective in treating hyperbilirubinemia, wherein the significantly lower dosage is therapeutically effective. In another embodiment, the invention embraces administration of low doses of stannsoporfin in order to reduce serum bilirubin levels. In another embodiment, the invention embraces administration of low doses of stannsoporfin to infants to reduce the need for an exchange transfusion.

In one embodiment, the invention embraces administration of relatively low doses of stannsoporfin to infants to treat hyperbilirubinemia. In one embodiment, the dosage used is 0.75 mg/kg birthweight, or about 0.75 mg/kg birthweight (1.0 umol/kg birthweight or about 1.0 umol kg birthweight). In another embodiment, the dosage used is 1.0 mg/kg birthweight, or about 1.0 mg/kg birthweight (1.33 umol/kg birthweight or about 1.33 umol kg birthweight). In another embodiment, the dosage used is 1.25 mg/kg birthweight, or about 1.25 mg/kg birthweight (1.67 umol/kg birthweight or about 1.67 umol kg birthweight). In another embodiment, the dosage used is 1.5 mg/kg birthweight, or about 1.5 mg/kg birthweight (2.0 umol/kg birthweight or about 2.0 umol kg birthweight). In another embodiment, the dosage used is 1.75 mg/kg birthweight, or about 1.75 mg/kg birthweight (2.33 umol/kg birthweight or about 2.33 umol kg birthweight). In another embodiment, the dosage used is 2.0 mg/kg birthweight, or about 2.0 mg/kg birthweight (2.67 umol/kg birthweight or about 2.67 umol kg birthweight). In another embodiment, the dosage used is 2.25 mg/kg birthweight, or about 2.25 mg/kg birthweight (3.0 umol/kg birthweight or about 3.0 umol kg birthweight). In another embodiment, the dosage used is 2.5 mg/kg birthweight, or about 2.5 mg/kg birthweight (3.33 umol/kg birthweight or about 3.33 umol kg birthweight). In another embodiment, the dosage used is 2.75 mg/kg birthweight, or about 2.75 mg/kg birthweight (3.67 umol/kg birthweight or about 3.67 umol kg birthweight). In another embodiment, the dosage used is 3.0 mg/kg birthweight, or about 3.0 mg/kg birthweight (4.0 umol/kg birthweight or about 4.0 umol kg birthweight).

In one embodiment, the invention embraces administration of relatively low doses of stannsoporfin to infants to treat hyperbilirubinemia. In one embodiment, the dosage used is 0.75 mg/kg bodyweight, or about 0.75 mg/kg bodyweight (1.0 umol/kg bodyweight or about 1.0 umol kg bodyweight). In another embodiment, the dosage used is 1.0 mg/kg bodyweight, or about 1.0 mg/kg bodyweight (1.33 umol/kg bodyweight or about 1.33 umol kg bodyweight). In another embodiment, the dosage used is 1.25 mg/kg bodyweight, or about 1.25 mg/kg bodyweight (1.67 umol/kg bodyweight or about 1.67 umol kg bodyweight). In another embodiment, the dosage used is 1.5 mg/kg bodyweight, or about 1.5 mg/kg bodyweight (2.0 umol/kg bodyweight or about 2.0 umol kg bodyweight). In another embodiment, the dosage used is 1.75 mg/kg bodyweight, or about 1.75 mg/kg bodyweight (2.33 umol/kg bodyweight or about 2.33 umol kg bodyweight). In another embodiment, the dosage used is 2.0 mg/kg bodyweight, or about 2.0 mg/kg bodyweight (2.67 umol/kg bodyweight or about 2.67 umol kg bodyweight). In another embodiment, the dosage used is 2.25 mg/kg bodyweight, or about 2.25 mg/kg bodyweight (3.0 umol/kg bodyweight or about 3.0 umol kg bodyweight). In another embodiment, the dosage used is 2.5 mg/kg bodyweight, or about 2.5 mg/kg bodyweight (3.33 umol/kg bodyweight or about 3.33 umol kg bodyweight). In another embodiment, the dosage used is 2.75 mg/kg bodyweight, or about 2.75 mg/kg bodyweight (3.67 umol/kg bodyweight or about 3.67 umol kg bodyweight). In another embodiment, the dosage used is 3.0 mg/kg bodyweight, or about 3.0 mg/kg bodyweight (4.0 umol/kg bodyweight or about 4.0 umol kg bodyweight).

In another embodiment, the low dose is within a range of about 0.75 to 3.0 mg/kg birthweight. In another embodiment, the low dose is within a range of about 0.75 to 2.75 mg/kg birthweight. In another embodiment, the low dose is within a range of about 0.75 to 2.5 mg/kg birthweight. In another embodiment, the low dose is with a range of about 0.75 to 2.25 mg/kg birthweight. In another embodiment, the low dose is with a range of about 1.0 to 2.25 mg/kg birthweight. In another embodiment, the low dose is with a range of about 1.0 to 2.0 mg/kg birthweight. In another embodiment, the low dose is with a range of about 1.25 to 2.0 mg/kg birthweight. In another embodiment, the low dose is with a range of about 1.25 to 1.75 mg/kg birthweight. In another embodiment, the low dose is with a range of about 1.25 to 1.5 mg/kg birthweight. In another embodiment, the low dose is with a range of about 1.5 to 1.75 mg/kg birthweight.

In another embodiment, the low dose is within a range of about 0.75 to 3.0 mg/kg birthweight. In another embodiment, the low dose is within a range of about 1.0 to 3.0 mg/kg birthweight. In another embodiment, the low dose is within a range of about 1.25 to 3.0 mg/kg birthweight. In another embodiment, the low dose is with a range of about 1.5 to 3.0 mg/kg birthweight. In another embodiment, the low dose is with a range of about 1.75 to 3.0 mg/kg birthweight. In another embodiment, the low dose is with a range of about 2.0 to 3.0 mg/kg birthweight. In another embodiment, the low dose is with a range of about 2.25 to 3.0 mg/kg birthweight. In another embodiment, the low dose is with a range of about 2.55 to 3.0 mg/kg birthweight. In another embodiment, the low dose is with a range of about 2.75 to 3.0 mg/kg birthweight.

In another embodiment, the low dose is within a range of about 0.75 to 3.0 mg/kg bodyweight. In another embodiment, the low dose is within a range of about 0.75 to 2.75 mg/kg bodyweight. In another embodiment, the low dose is within a range of about 0.75 to 2.5 mg/kg bodyweight. In another embodiment, the low dose is with a range of about 0.75 to 2.25 mg/kg bodyweight. In another embodiment, the low dose is with a range of about 1.0 to 2.25 mg/kg bodyweight. In another embodiment, the low dose is with a range of about 1.0 to 2.0 mg/kg bodyweight. In another embodiment, the low dose is with a range of about 1.25 to 2.0 mg/kg bodyweight. In another embodiment, the low dose is with a range of about 1.25 to 1.75 mg/kg bodyweight. In another embodiment, the low dose is with a range of about 1.25 to 1.5 mg/kg bodyweight. In another embodiment, the low dose is with a range of about 1.5 to 1.75 mg/kg bodyweight.

In another embodiment, the low dose is within a range of about 0.75 to 3.0 mg/kg bodyweight. In another embodiment, the low dose is within a range of about 1.0 to 3.0 mg/kg bodyweight. In another embodiment, the low dose is within a range of about 1.25 to 3.0 mg/kg bodyweight. In another embodiment, the low dose is with a range of about 1.5 to 3.0 mg/kg bodyweight. In another embodiment, the low dose is with a range of about 1.75 to 3.0 mg/kg bodyweight. In another embodiment, the low dose is with a range of about 2.0 to 3.0 mg/kg bodyweight. In another embodiment, the low dose is with a range of about 2.25 to 3.0 mg/kg bodyweight. In another embodiment, the low dose is with a range of about 2.55 to 3.0 mg/kg bodyweight. In another embodiment, the low dose is with a range of about 2.75 to 3.0 mg/kg bodyweight.

In one embodiment, the low dose of stannsoporfin is administered to a term baby. In another embodiment, the low dose of stannsoporfin is administered to a near-term baby. In another embodiment, the lose dose of stannsoporfin is administered to a baby of at least about 35 weeks gestational age. In another embodiment, the low dose of stannsoporfin is administered to a baby of at least about 38 weeks gestational age. In another embodiment, the low dose of stannsoporfin is administered to a baby of at least about 39 weeks gestational age. In another embodiment, the low dose of stannsoporfin is administered to a baby of at least about 40 weeks gestational age.

In another embodiment, the low dose of stannsoporfin is administered to a baby with a birthweight or bodyweight above 2000 grams, or above about 2000 grams. In another embodiment, the low dose of stannsoporfin is administered to a baby with a birthweight or bodyweight above 2500 grams, or above about 2500 grams. In another embodiment, the low dose of stannsoporfin is administered to a baby with a birthweight or bodyweight above 3000 grams, or above about 3000 grams.

In one embodiment, the low dose of stannsoporfin is not administered at birth. In another embodiment, the low dose of stannsoporfin is administered at least about 12 hours after birth. In another embodiment, the low dose of stannsoporfin is administered at least about 18 hours after birth. In another embodiment, the low dose of stannsoporfin is administered at least about 24 hours after birth. In another embodiment, the low dose of stannsoporfin is administered at least about 30 hours after birth. In another embodiment, the low dose of stannsoporfin is administered at least about 36 hours after birth. In another embodiment, the low dose of stannsoporfin is administered at least about 42 hours after birth. In another embodiment, the low dose of stannsoporfin is administered at least about 48 hours after birth. In another embodiment, the low dose of stannsoporfin is administered at least about 60 hours after birth. In another embodiment, the low dose of stannsoporfin is administered at least about 72 hours after birth. In another embodiment, the low dose of stannsoporfin is administered at least about 96 hours after birth. In another embodiment, the low dose of stannsoporfin is administered at least about 5 days after birth. In another embodiment, the low dose of stannsoporfin is administered at least about 6 days after birth. In another embodiment, the low dose of stannsoporfin is administered at least about 7 days after birth.

In one embodiment, the low dose of stannsoporfin is not administered at birth. In another embodiment, the low dose of stannsoporfin is administered when the infant's postnatal age and serum bilirubin level place the infant in the high risk zone as defined in the American Academy of Pediatrics Clinical Practice Guideline entitled “Management of Hyperbilirubinemia in the Newborn Infant 35 or More Weeks of Gestation,” Pediatrics 114:297-316 (2004). In another embodiment, the low dose of stannsoporfin is administered when the infant's postnatal age and serum bilirubin level place the infant in the high intermediate risk zone as defined in the previously mentioned American Academy of Pediatrics Clinical Practice Guideline. In another embodiment, the low dose of stannsoporfin is administered when the infant's postnatal age and serum bilirubin level place the infant in the low intermediate risk zone as defined in the previously mentioned American Academy of Pediatrics Clinical Practice Guideline.

In another embodiment, the low dose of stannsoporfin is administered when an infant's serum bilirubin level is higher than about 7 mg/dL and the infant is about 24 hours old, or at least about 24 hours old. In another embodiment, the low dose of stannsoporfin is administered when an infant's serum bilirubin level is higher than about 13 mg/dL and the infant is about 48 hours old, or at least about 48 hours old. In another embodiment, the low dose of stannsoporfin is administered when an infant's serum bilirubin level is higher than about 16 mg/dL and the infant is about 72 hours old, or at least about 72 hours old.

In another embodiment, the low dose of stannsoporfin is administered when an infant's serum bilirubin level is higher than about 6 mg/dL and the infant is about 24 hours old, or at least about 24 hours old. In another embodiment, the low dose of stannsoporfin is administered when an infant's serum bilirubin level is higher than about 11 mg/dL and the infant is about 48 hours old, or at least about 48 hours old. In another embodiment, the low dose of stannsoporfin is administered when an infant's serum bilirubin level is higher than about 13 mg/dL and the infant is about 72 hours old, or at least about 72 hours old.

In another embodiment, the low dose of stannsoporfin is administered when an infant's serum bilirubin level is higher than about 5 mg/dL and the infant is about 24 hours old, or at least about 24 hours old. In another embodiment, the low dose of stannsoporfin is administered when an infant's serum bilirubin level is higher than about 8 mg/dL and the infant is about 48 hours old, or at least about 48 hours old. In another embodiment, the low dose of stannsoporfin is administered when an infant's serum bilirubin level is higher than about 11 mg/dL and the infant is about 72 hours old, or at least about 72 hours old.

In another embodiment, the low dose of stannsoporfin is administered when an infant's serum bilirubin level is greater than about 7 mg/dL. In another embodiment, the low dose of stannsoporfin is administered when an infant's serum bilirubin level is greater than about 10 mg/dL. In another embodiment, the low dose of stannsoporfin is administered when an infant's serum bilirubin level is greater than about 15 mg/dL. In another embodiment, the low dose of stannsoporfin is administered when an infant's serum bilirubin level is greater than about 20 mg/dL. In another embodiment, the low dose of stannsoporfin is administered when an infant's serum bilirubin level is greater than about 7 mg/dL and the infant is about 24 to 60 hours old, or at least about 24 to 60 hours old. In another embodiment, the low dose of stannsoporfin is administered when an infant's serum bilirubin level is greater than about 10 mg/dL and the infant is about 24 to 60 hours old, or at least about 24 to 60 hours old. In another embodiment, the low dose of stannsoporfin is administered when an infant's serum bilirubin level is greater than about 15 mg/dL and the infant is about 60 to 84 hours old, or at least about 60 to 84 hours old. In another embodiment, the low dose of stannsoporfin is administered when an infant's serum bilirubin level is greater than about 20 mg/dL and the infant is about 60 to 84 hours old, or at least about 60 to 84 hours old.

In any of the above embodiments, the administration of stannsoporfin can be combined with additional therapy to lower bilirubin levels, where such therapy can be provided before, during, or after stannsoporfin administration. Such therapies include, but are not limited to, phototherapy and exchange transfusion.

### BRIEF DESCRIPTION OF THE FIGURES

FIG. 1 depicts the effect of a 0.75 mg/kg birthweight dose of stannsoporfin (filled circle) and a 1.5 mg/kg birthweight dose of stannsoporfin (open diamond) on total serum bilirubin levels, compared to placebo (open circle) for treatment of hyperbilirubinemia. Babies who underwent exchange transfusion are excluded from all groups (note that all groups underwent phototherapy).

FIG. 2 depicts data for babies in the placebo group (open triangle) who received exchange transfusions, versus the stannsoporfin-treated babies who did not receive exchange transfusions, the 0.75 mg/kg dose group (open circles), and the 1.5 mg/kg dose group (filled circle), as a percentage of bilirubin levels. Note again that all infants, including those in the placebo group, received phototherapy.

### DETAILED DESCRIPTION OF THE INVENTION

Stannsoporfin (tin (IV) mesoporphyrin IX dichloride; Chemical Abstracts Registry Number 106344-20-1) is also known by the trade name Stanate®, which is a registered trademark of InfaCare Pharmaceutical Corp., Plymouth Meeting, Pa. Stannsoporfin has the following structure:

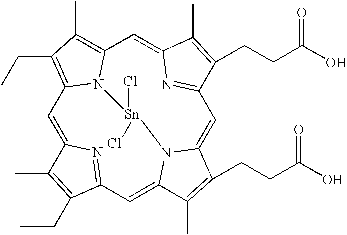

having molecular formula C₃₄H₃₆Cl₂N₄O₄Sn and molecular weight 754.29.

The abbreviation “kg-biw” indicates “kilograms of birthweight” and refers to the weight of the infant at birth. The abbreviation “kg-bow” indicates “kilograms bodyweight” and refers to the weight of the infant at a specific point in time (for example, at the time of administration of stannsoporfin).

By “low-dose” or “low-dosage” is meant a dosage of stannsoporfin of about 3.0 mg/kg or less, either with respect to kg birthweight or kg bodyweight. While the dosage of stannsoporfin to be used is typically calculated with respect to the birthweight of the infant, the bodyweight of the infant at the time of administration can also be used to calculate the amount of stannsoporfin for administration.

“dL” or “dl” is an abbreviation for deciliter.

A “term” baby refers to a baby of at least approximately 38-40 weeks gestational age. A “near-term” baby refers to a baby of approximately 35 weeks to approximately 37 weeks and six days gestational age.

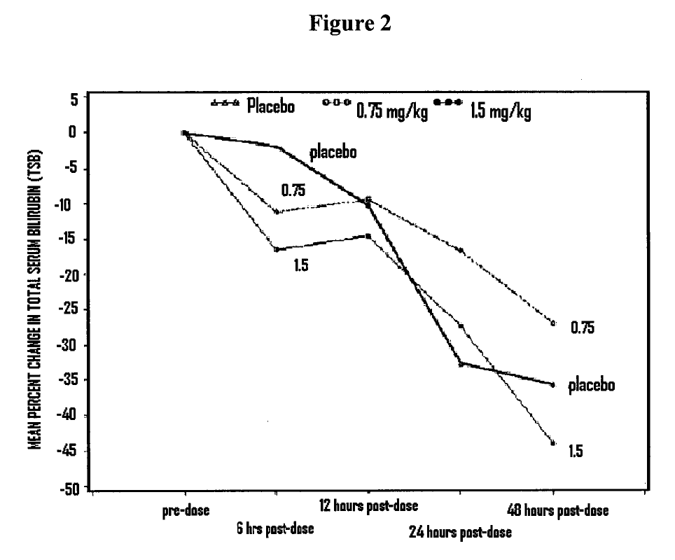

“High risk zone,” “high intermediate risk zone,” “low intermediate risk zone,” and “low risk zone” as used herein are as defined in the American Academy of Pediatrics Clinical Practice Guideline of the Subcommittee on Hyperbilirubinemia, as published in “Management of Hyperbilirubinemia in the Newborn Infant 35 or More Weeks of Gestation,” Pediatrics 114:297-316 (2004). That publication is hereby incorporated by reference herein in its entirety, particularly FIG. 2 at page 301 where the designations of high risk zone, high intermediate risk zone, low intermediate risk zone, and low risk zone are provided in terms of an infant's postnatal age and serum bilirubin level.

Other guidelines for treatment of hyperbilirubinemia are extant, in addition to the previously mentioned American Academy of Pediatrics guideline, and the low dose treatment of stannsoporfin can be used for infants at risk according to those guidelines as well. (See, for example, Porter, M. L. and Dennis, B. L., “Hyperbilirubinemia in the Term Newborn,” Am. Fam. Physician 65:599-606, 613-4 (2002).)

### Use of Low-Dose Stannsoporfin in Treatment of Infant Hyperbilirubinemia

Previous work on clinical applications of stannsoporfin for treatment of infant hyperbilirubinemia provided support for use of a dosage of 4.5 mg/kg, as suggested by the studies by Valaes et al., Pediatrics 93:1-11 (1994) and Kappas et al., Pediatrics, 95:468-474 (1995); the Valaes 1994 study was limited to pre-term (premature) infants. The current invention is based on the discovery that stannsoporfin is able to control severe hyperbilirubinemia and reduce the need for exchange transfusion in term infants when administered at much lower dosages, including (but not limited to) dosages such as 0.75 mg/kg and 1.5 mg/kg. In addition, the low dose of stannsoporfin need not be administered immediately after birth or shortly thereafter (within about 0 to 6 hours of age) to be effective, but rather can be administered after monitoring the infant for some time, such as 1, 2, 3, 4, 5, or 6 days after birth. That is, a physician need not administer the stannsoporfin prophylactically, but can administer the low dose of stannsoporfin only when necessary for a therapeutic effect.

Establishing that a lower dosage of stannsoporfin can be effective for treatment of hyperbilirubinemia is important for many reasons. While stannsoporfin has relatively few side effects, reducing the dosage can reduce side effects even further. Using the lowest therapeutically effective dosage of a pharmaceutical is highly advantageous for this unique patient population. Using lower dosages of a pharmaceutical can also enable usage of lower volumes for injection, which can help reduce discomfort to the infant. Additionally, for countries where the primary cost of a pharmaceutical is the cost to manufacture the drug (such as in developing countries, where drugs are often provided at little or no cost to the patient on a compassionate basis), a lower dosage means that more patients can be treated at the same cost.

Use of low-dose stannsoporfin in hyperbilirubinemia can reduce the need for exchange transfusions, which is highly desirable. Use of low-dose stannsoporfin in hyperbilirubinemia can also prevent “rebound” hyperbilirubinemia, as shown below in the example.

### Pharmaceutical Considerations

The stannsoporfin can be provided and administered as a single dose unit (unit dosage). Multiple doses of stannsoporfin, which, when added together, contain the same amount of stannsoporfin as a single low dose, can also be used. Stannsoporfin is typically supplied at a concentration of 20 mg/ml; other concentrations can be used, provided that the volume to be administered to an infant is not excessive.

The stannsoporfin is preferably provided in a solution which can be buffered to maintain a suitable pH. Buffers which can be used include phosphate, citrate, gluconate, lactate, tartrate, glycinate, glycylglycinate, bicarbonate, carbonate, maleate, or acetate, with sodium, potassium, magnesium, calcium, or aluminum present as the cation. Histidine and imidazole can also be used as buffers. Phosphate buffers are preferred, particularly sodium phosphate buffer. Buffers must be pharmaceutically acceptable for use as an injectable agent in neonates.

The pH of the solution for administration is preferably between about 7.0 to 8.0, more preferably about 7.2 to 7.9, still more preferably about 7.4. The osmolarity of the solution is preferably at or near physiological osmolarity; a preferred range is between about 280 mOsm/L and 310 mOsm/L.

Stannsoporfin is preferably administered by injection, more preferably by intramuscular injection.

The following example is intended to illustrate the invention, and is not intended to limit the invention in any manner.

### EXAMPLE

### Effect of Low-Dose Administration of Stannsoporfin

Low-dose administration of stannsoporfin was tested in a group of infants in Hanoi, Vietnam and Michigan, United States. The known and unknown risks of the proposed study intervention and the potential for direct and indirect benefit were discussed with each subject's parents or guardians. Written informed consent was obtained from a parent or guardian of each subject before enrollment in the study. The study protocol was conducted under the guidelines of current Good Clinical Practice as promulgated by the United States Food and Drug Administration and international regulatory bodies. The study was reviewed and approved by the Institutional Review Board of the William Beaumont Hospital, Royal Oak, Mich., and the Vietnamese Ministry of Health and the Ethics Review Board of the National Hospital for Pediatrics of Hanoi.

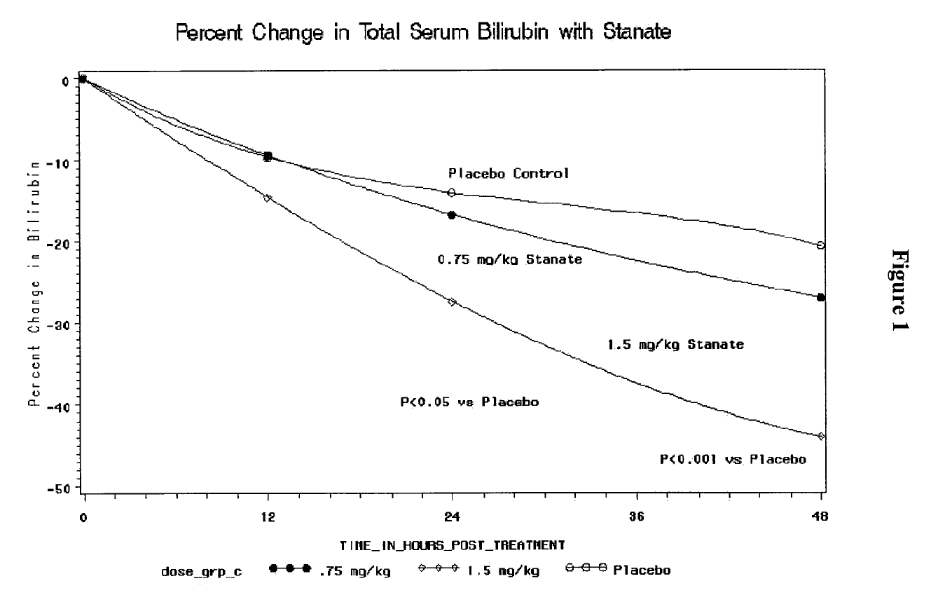

All infants received phototherapy to treat hyperbilirubinemia; thus, the group designated as “placebo” were treated with phototherapy, as were the other groups. The placebo group had 18 babies total. In the placebo group, 9 babies received exchange transfusions (1 baby received an exchange transfusion before treatment; 9 babies received exchange transfusion after treatment, including the one who received an exchange transfusion before treatment); 9 babies did not receive an exchange transfusion. The 0.75 mg/kg stannsoporfin group had 19 babies total. In the 0.75 mg/kg group, 3 babies total received exchange transfusions (2 babies received an exchange transfusion before treatment; 1 baby received an exchange transfusion after treatment); 16 babies did not receive an exchange transfusion. The 1.5 mg/kg stannsoporfin group had 18 babies total. In the 1.5 mg/kg group, 2 babies total received exchange transfusions (no babies received an exchange transfusion before treatment; 2 babies received an exchange transfusion after treatment); 16 babies did not receive exchange transfusion. Treatment refers to either placebo injection or stannsoporfin injection; as previously indicated, all babies received phototherapy. The placebo used was normal physiological saline. Stannsoporfin was administered via intramuscular injection in sodium phosphate buffer at a concentration of 20 mg/ml. A vial containing 1.5 ml was used, where a dose based on birthweight of the infant and whether a 0.75 mg/kg or 1.5 mg/kg dose was withdrawn. (For example, a 5 kg birthweight baby treated at a dose of 1.5 mg/kg would receive 7.5 mg of stannsoporfin; thus, a volume of 0.375 ml from the 20 mg/ml solution would be used to provide this dose.) In the majority of cases, the infants were between 4 and 7 days of age at the time of either stannsoporfin administration or exchange transfusion. The results are shown in FIG. 1 and FIG. 2; infants receiving stannsoporfin injections who required exchange transfusions before or after treatment are not included in the data presented in FIG. 1 for any group, while babies in the stannsoporfin groups who required exchange transfusion are excluded from FIG. 2 and only babies in the placebo group that did receive exchange transfusion are included in FIG. 2.

FIG. 1 shows the comparative results of stannsoporfin treatment at 0.75 mg/kg (filled circles) and 1.5 mg/kg (open diamonds) to placebo treatment (open circles); babies who required exchange transfusions are excluded from the data set used in FIG. 1. Both doses of stannsoporfin showed a greater reduction of serum bilirubin than the placebo group (phototherapy only). The 1.5 mg/kg result had a significance value P<0.05 versus placebo at 24 hours and P<0.001 versus placebo at 48 hours. The data demonstrate that low dosages of stannsoporfin are effective at controlling bilirubin levels in infants with severely elevated bilirubin levels.

FIG. 2 depicts the data in the form of percentage reduction in starting bilirubin levels for the placebo injection for babies that received exchange transfusion, the 0.75 mg/kg birthweight stannsoporfin group that did not receive exchange transfusion, and the 1.5 mg/kg birthweight stannsoporfin group that did not receive exchange transfusion; thus the graph essentially compares exchange transfusion to 0.75 mg/kg and 1.5 mg/kg stannsoporfin treatment. Note that all babies received phototherapy. The placebo group is designated by open triangles; the 0.75 mg/kg group by open circles/open squares, and the 1.5 mg/kg group by filled circles/filled squares. Both stannsoporfin groups showed substantial decrease in serum bilirubin; at 1.5 mg/kg, the decrease in serum bilirubin was greater than that for the exchange transfusion group at 3 of the 4 post-treatment data points, and the 1.5 mg/kg injection caused bilirubin to decline at a rate similar to that caused by exchange transfusion for the period between 12 and 24 hours. (A 6-hour time point is included in this graph, which shows a dip at 6 hours below the 12-hour bilirubin level; this point is not included in FIG. 1.)

The following table indicates the number of babies requiring exchange transfusion in the initial groups. Administration of the low-dose stannsoporfin significantly decreased the need for exchange transfusion. Given that the low dose stannsoporfin treatment was effective in these cases of severe hyperbilirubinemia, use of the low dose stannsoporfin treatment in less severe cases should also be of great utility.

| | control | 0.75 mg/kg | 1.5 mg/kg |
|---|---|---|---|
| | N = 18 | N = 19 | N = 18 |
| # babies | 18 | 19 | 18 |
| enrolled | | | |
| # transfused pts | 9 | 3 | 2 |
| % requiring | 50% | 16% | 11% |
| transfusions | | | |

Regarding side effects, there were no statistically significant differences among the three treatment groups in the incidence, severity, seriousness, and causality of adverse events. Greater than 60% of the infants in each treatment group experienced at least one adverse event, all of which were mild or moderate in severity, with one exception, noted below. None of the adverse events were considered probably or definitely related to the study treatment. One (5.6%) infant in the placebo treatment group experienced a serious adverse event, moderate meningitis, which was non-treatment-related and which resolved within two months.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of treating hyperbilirubinemia in an infant of at least about 38 weeks gestational age, comprising:
administering a low dose of stannsoporfin to an infant in need thereof.

2. The method of claim 1, wherein the low dose of stannsoporfin is about 0.75 mg/kg birthweight.

3. The method of claim 1, wherein the low dose of stannsoporfin is about 0.75 mg/kg bodyweight.

4. The method of claim 1, wherein the low dose of stannsoporfin is about 1.5 mg/kg birthweight.

5. The method of claim 1, wherein the low dose of stannsoporfin is about 1.5 mg/kg bodyweight.

6. The method of claim 1, wherein the low dose of stannsoporfin is about 2.25 mg/kg birthweight.

7. The method of claim 1, wherein the low dose of stannsoporfin is about 2.25 mg/kg bodyweight.

8. The method of claim 1, wherein the low dose of stannsoporfin is about 3.0 mg/kg birthweight.

9. The method of claim 1, wherein the low dose of stannsoporfin is about 3.0 mg/kg bodyweight.

10. The method of claim 1, wherein the low dose of stannsoporfin is within about 0.75 to 3.0 mg/kg birthweight.

11. The method of claim 1, wherein the low dose of stannsoporfin is within about 0.75 to 3.0 mg/kg bodyweight.

12. The method of claim 1, wherein the low dose of stannsoporfin is within about 1.0 to 2.0 mg/kg birthweight.

13. The method of claim 1, wherein the low dose of stannsoporfin is within about 1.0 to 2.0 mg/kg bodyweight.

14. The method of claim 1, wherein the low dose of stannsoporfin is administered to the infant at least about 24 hours after birth.

15. The method of claim 1, wherein the infant's postnatal age and serum bilirubin level places the infant in the high risk zone of hyperbilirubinemia.

16. The method of claim 1, wherein the infant's postnatal age and serum bilirubin level places the infant in the high intermediate risk zone of hyperbilirubinemia.

17. The method of claim 1, wherein the infant's postnatal age and serum bilirubin level places the infant in the low intermediate risk zone of hyperbilirubinemia.

18. The method of claim 1, wherein the infant is of at least about 39 weeks gestational age.

19. The method of claim 1, wherein the infant is of at least about 40 weeks gestational age.
